(19) United States
(12) Patent Application Publication
(10) Pub. No.: US 2026/0240659 A1
(43) Pub. Date: Aug. 20, 2026

(54) Components for Use in a Universal Shoulder Prosthesis System

(71) Applicant: Raphael S.F. Longobardi, LLC, Old Tappan, NJ (US)

(72) Inventor: Raphael S.F. Longobardi, Old Tappan, NJ (US)

(21) Appl. No.: 18/995,036

(22) PCT Filed: Jul. 21, 2023
(86) PCT No.: PCT/US2023/028397

§ 371 Date: Jan. 15, 2025

Related U.S. Application Data
(60) Provisional application No. 63/391,306, filed on Jul. 21, 2022.

(51) Int. Cl. A61F 2/40; A61F 2/30

(52) U.S. Cl. CPC A61F 2/4014; A61F 2002/30604; A61F 2002/4022

(57) ABSTRACT

A universal shoulder prosthesis system includes and one or more modular components that are exchangeable between ta-TSR and r-TSR. In at least one embodiment, the humeral components that contact the glenoid components include polyethylene while the glenoid components that contact the humeral components are all metal. An example of the modular components for ta-TSR are a glenoid modular component (200) having a concave cavity (212) and a humeral modular component (300) includes an outer shell (310) and an inner head (320) that fits into the outer shell (310). An example of the modular components for r-TSR are a glenosphere (516), which is the glenoid modular component (500), having a 10 degree inferior tilt built-in and a humeral cup (600), which is the humeral modular component, with a replaceable shell (650) that partially sits within a base (610), which optionally may include metal.

This PCT application claims priority to U.S. Provisional Patent App. No. 63/391,306, filed on Jul. 21, 2022, which is incorporated by reference.

### I. FIELD OF THE INVENTION

The invention relates to a modular shoulder prosthesis system and/or individual system components that provide for flexibility in shoulder replacements and allows for a more efficient switch for a patient between a traditional anatomic Total Shoulder Replacement (ta-TSR) to a reverse Total Shoulder Replacement (r-TSR). In at least one embodiment, the system also, or alternatively, provides for a modular adaptation for the glenoid and/or humeral sides in a ta-TSR and/or r-TSR.

### II. BACKGROUND OF THE INVENTION

TSRs have evolved over the last 75 years, with the greatest degree of its evolution occurring within the past 25 years. The understanding of the complexity of the shoulder has resulted in the ability to better treat the multiple conditions that afflict the shoulder. Glenohumeral arthritis ranges from simple to complex due to etiology and deformity. Post traumatic glenohumeral arthritis, along with the deformity of both the glenoid and humeral head present challenges for the shoulder arthroplasty surgeon. Similarly, the problem of rotator cuff deficiency and rotator cuff arthropathy has resulted in the development of treatment and prosthetic designs specific to address the loss of the main motors of the shoulder.

Currently, there are two types of TSR-traditional anatomic total shoulder replacement (ta-TSR) and reverse total shoulder replacement (r-TSR). Ta-TSR utilizes resurfacing of the humeral head and glenoid in the setting of an intact and functioning rotator cuff. Glenohumeral arthritis has been treated with ta-TSR, the current gold standard being the resurfacing of the humeral head with a stemmed or metaphyseal component along with a replacement of the humeral head articular portion with a Cobalt-Chromium (Co—Cr) implant. Modularity of the humeral components allows for appropriate sizing of the head in diameter and thickness to match the resected articular surface of the patient.

To revise from the ta-TSR to r-TSR often requires removal of the glenoid component and reconstruction of the glenoid bone stock. Ta-TSR utilize all polyethylene glenoid components which become the typical point of failure for the ta-TSR. R-TSR utilizes a porous in-growth metal design with locking screws for glenoid fixation.

The most common cause of failure of the ta-TSR is due to glenoid loosening secondary to rotator cuff failure/tear. The resulting superior migration of the humeral head, with concomitant change in the center of rotation (C.O.R.) from rotator cuff failure produces edge loading of the glenoid component. This asymmetric mechanical loading results in rocking and loosening of the polyethylene prosthesis from the cement and bone of the glenoid.

R-TSR evolved from the specific abnormal mechanics of the rotator cuff deficient shoulder, as previously described. In the rotator deficient condition, the deltoid muscle becomes the predominant motor, but in an inefficient manner. The deltoid muscle contraction functions to result in “hinged abduction” of the humeral head/humeral shaft. The humeral head and greater tuberosity lever on the undersurface of the acromion and superior portion of the glenoid. Ta-TSR is contra-indicated in the setting of rotator cuff deficiency, due to the known catastrophic results to the glenoid component.

The development of the r-TSR addresses the rotator cuff deficient, painful arthritic shoulder. The design of r-TSR is to maximize deltoid fiber length to allow more efficient contraction and function of the deltoid in elevation of the arm. The prosthetic components are designed to change the C.O.R. to one that is more inferior and medial to the native joint.

R-TSRs already have asymmetric, higher shear and higher loading of the glenoid component, called the glenosphere and baseplate construct. Despite these greater loads, in growth metal baseplates with locking screws are not the cause of failure, due to the excellent bone incorporation and stability.

The r-TSR has a different humeral component design as well. Where the ta-TSR has a humeral head, the r-TSR had a glenosphere attached to the glenoid. The humerus had a stemmed component but attached to the top is a humeral cup to articulate with the glenosphere. The modularity of components, specifically glenosphere sizing and humeral cup sizing, allow for multiple permutations to achieve the most successful and stable construct.

### III. SUMMARY OF THE INVENTION

In at least one TSR embodiment, there is anticipated to be a decrease in rotator cuff failure and glenoid loosening. In at least one reverse TSR embodiment, there is anticipated to be improved stability and less notching. In at least one embodiment, it is anticipated there will be a general decrease in complications while reducing patient recovery time and diminished operative time (leading to reduction in costs) when going from a previous TSR to r-TSR from needing to revise only articulating modular components and not the baseplates.

In at least one embodiment, the baseplate is universal to the glenoid and humeral sides and configured for receiving different modular articulating components. In at least one embodiment, the baseplate includes a central reverse Morse cavity that accommodates all articulating taper components. In at least one further embodiment, the baseplate includes a screw fixation with a central compression screw with peripheral variable angle locking screws to improve humeral and glenoid fixation while mitigating loosening. The use of a universal baseplate leads to a smaller inventory and fewer instruments as it can be used on both the glenoid side and the humeral side. In at least one embodiment to the other embodiments in this paragraph, the baseplate includes a fixation attachment hole or chamber to receive a screw at least partially passing through a modular component to further secure the modular component to the baseplate.

In at least one ta-TSR embodiment, there is a glenoid modular component and/or a humeral modular component that are mountable onto respective baseplates. The glenoid modular component includes a concave surface for receiving in it a domed surface of the humeral modular component. In a further embodiment, the glenoid modular component is elliptical or oval-shaped with the concave surface centrally located on the surface configured to face the humeral modular component. The glenoid modular component is composed of various metals used within joint arthroplasty, and in a further embodiment the materials used can include titanium, cobalt-chrome and titanium nitride. In at least one embodiment, the humeral modular component includes an inner head that fits into a domed shell that is configured to motive relative to the concave surface of the glenoid modular component. Examples of materials that may be used for the inner head include various metals including those that may be used for the glenoid modular component and additional examples includes titanium, cobalt-chrome and titanium nitride; while examples of materials that may be used for the shell include polyethylene and/or of other polyethylene fabrications.

In at least one r-TSR embodiment, there is a glenosphere and/or a humeral cup that are mountable onto respective baseplates and are examples of modular components. In at least one glenosphere embodiment, the glenosphere is composed of various metals, and in a further embodiment the materials used can include titanium, cobalt-chrome and titanium nitride. In at least one embodiment or a further embodiment, the glenosphere includes a built-in 10 degree of inferior angulation and/or a 3 mm offset creating an equivalent starting hole (4 mm inferior) to the implanted baseplate center. In at least one embodiment, an advantage is inferior notching mitigation as the glenosphere still sits at the inferior edge of the glenoid. In at least one embodiment, an advantage is obviating revision by mitigating the need to remove the baseplate and/or harvest local autograft improving surgical times and patient recovery. In a further embodiment to the other embodiments in this paragraph, the glenosphere includes a passageway with a setting surface, which may be beveled, for receiving a screw or bolt for further securing the glenosphere to the baseplate with a fixation attachment hole or chamber. In at least one humeral cup embodiment, the humeral cup includes a humeral base and a humeral shell that fits into the humeral base and is configured to provide a surface on which a glenosphere is able to move. The humeral shell may be made of various polyethylene fabrications.

The following modular component embodiments may be used in a modular shoulder prosthesis system having a baseplate with or without notches, attachment points, and/or fixation attachment hole.

In at least one embodiment, a glenoid modular component for r-TSR includes a base having an optional peripheral flange that extends down to fit over the baseplate when installed providing an interference fit; a glenosphere extending from said base with an inferior tilt of approximately 7 to approximately 10 degrees, 7 to 10 degrees, or approximately 10 degrees as measured from a reference axis that is perpendicular to said base with a minor axis that intersects the reference axis and the anterior and posterior sides of said glenosphere and a major axis that is perpendicular to the minor axis, said glenosphere optionally including a passageway passing therethrough and through said base and perpendicular to said base and offset along the major axis from a superior side of said glenosphere; and an optional plug extending from a surface of said base opposite said glenosphere and centered on the minor axis and oft center of the major axis. In a further embodiment, said glenosphere is approximately a three-quarters oblong sphere. In a further embodiment to any of the other embodiments in this paragraph, a majority of a mass of said glenosphere is on an inferior side of the minor axis, optionally configured to align the mass with a complementary humeral modular component. In a further embodiment to the first two embodiments, said glenosphere eccentrically located on said base relative to said plug. In a further embodiment to any of the previous embodiments in this paragraph, the posterior side and the anterior side of said glenosphere are substantially parallel to the reference axis over a majority of the respective height of said posterior side and said anterior side. In a further embodiment to any of the previous embodiments of this paragraph, said base, said plug and said glenosphere are integrally formed. In a further embodiment to the previous embodiments, said glenosphere having a 10 mm lateralization of the center of rotation (COR) with a diameter of 32 mm, 36 mm, or 40 mm or a 6 mm lateralization of the COR with a diameter of 32 mm, 36 mm, 40 mm, or 44 mm with a minus size of 4 mm. In a further embodiment to any of the previous passageway embodiments, said passageway includes a shoulder configured to engage a head of a fastening screw and a threaded section on a baseplate side of the shoulder. In a further embodiment to any of the passage embodiments, said passageway is located between the superior side of said glenosphere and one or both of the reference axis and the minor axis. In a further embodiment to any of the flange embodiments, said flange has a narrower width on the superior side increasing along the anterior and posterior sides to thicker width on the inferior side. In a further embodiment to any of the previous embodiments, said glenosphere includes or is made of Co—Cr or includes or is made of one or more metals.

In at least one embodiment, a humeral modular component for r-TSR including: a humeral cup base having a receiving cavity, an optional plug extending away from said base, and an optional flange extending away from a peripheral edge of said base, said flange configured to fit over the baseplate; and a humeral cup shell configured to be removably inserted into the receiving cavity of said humeral cup base, said shell including a curved cavity. In a further embodiment, said base includes a wall, which optionally may be cylindrical, extending up to define the receiving cavity and said wall optionally includes notches extending down from a top of the wall and/or a groove around interior bottom of said wall. In a further embodiment, said wall includes a beveled or rounded interior edge and/or a beveled or rounded exterior edge on its top. In a further embodiment to any of the previous embodiments in this paragraph, said shell includes a base with said curved cavity framed by a flat surface, and a wall extending down from a surface of said base opposite said curved cavity, said wall is inset in from an outer peripheral surface of said base. In a further embodiment, wherein said shell wall including a plurality of protrusions for engaging an optional groove of said humeral cup base, said protrusions are evenly spaced from each other. In a further embodiment to the previous two embodiments, each of said protrusions include a free end having a hook for engaging the groove of said humeral cup base, optionally said protrusions and/or said wall are sufficiently flexible to allow for insertion of the shell into said receiving cavity of said humeral cup base. In a further embodiment to the other shell embodiments, said flat surface of said base includes a beveled or rounded interior edge and/or a beveled or rounded exterior edge on its top; said flat surface is inclined from its exterior periphery towards said curved cavity; and/or the surface area of said curved cavity is greater than a surface area of said flat surface. In a further embodiment to any of the shell embodiments, said curved cavity has a variating surface with a non-uniform level of curvature over the surface of said curved cavity and/or said curved cavity is concave. In a further embodiment to any of the previous embodiments of this paragraph, said humeral cup base is made from Co—Cr and said shell includes or is made of polyethylene or a related articulating material or Vitamin E HDPE.

In at least one embodiment, a modular shoulder prosthesis system includes a glenoid baseplate, a humeral baseplate or humeral base, any of the glenoid modular embodiments for r-TSR described above, and any of the humeral modular components for r-TSR described above.

In at least one embodiment, a humeral modular component for ta-TSR including: an inner head having a base, a dome on said base, optionally said dome and said base are integrally formed, an optional plug extending away from said base; and a shell configured to fit over said inner head, said shell having a body having an exterior surface configured for engagement with a concave surface of a glenoid modular component, said body defining an interior cavity configured to receive said dome, and an interiorly facing engagement means for attachment of said shell to said inner head where said engagement means is in the interior cavity, and wherein said engagement means includes a ring protrusion that engages a channel around a peripheral lower edge of said base, or said engagement means includes a channel that engages a ring protrusion around a peripheral lower edge of said base, and/or wherein said a lower exterior periphery of said exterior surface is beveled or rounded. In a further embodiment, said dome and said interior cavity have complementary surfaces. In a further embodiment to either of the previous two embodiments, said ring protrusion is bonded to said channel to affix said outer shell to said inner head. In a further embodiment to any of the other embodiments of this paragraph, said base further includes a shoulder above said channel and a lower shoulder below said channel that together said shoulders define a groove or said engagement means further includes a shoulder above said channel and a lower shoulder below said channel that together said shoulders define a groove. In a further embodiment to any of the other embodiments of this paragraph, said ring protrusion and said channel have complementary shapes configured to allow for rotation of said shell relative to said inner head; said ring protrusion and said channel have an approximate semi-circular cross-section or a partial elliptical shape; and/or said ring protrusion and said channel have complementary curved surfaces when viewed in cross-section formed when a plane is perpendicular to said base and passing through an axial center of said humeral modular component. In a further embodiment to any of the other embodiments of this paragraph. said body having a flange configured to extend below said base of said inner head and overlap with the baseplate on which said humeral modular component is implanted. In a further embodiment to any of the other embodiments of this paragraph, said cavity includes a beveled surface proximate to its opening that is wider than said base and/or a cylindrical opening at its opening that transitions into the beveled surface. In a further embodiment to any of the other embodiments of this paragraph, said body has a thickness along it center axis that is aligned with said inner head than a lateral thickness around its lower end. In a further embodiment to any of the previous embodiments of this paragraph, said inner head includes cobalt-chromium and said shell includes a high-density polyethylene; said inner head includes Ti6Al4V, a titanium-aluminum alloy, or cobalt-chromium and/or said shell includes UHMWPE-highly crosslinked, a Vitamin E doped polyethylene, or Vitamin E HDPE; or said shell includes or is made of polyethylene.

In at least one embodiment, a glenoid modular component for ta-TSR including: a base having a cavity configured to receive a hemispheric humeral head or a prosthetic replacement of same, an optional plug extending from a surface of said base opposite said cavity, and an optional flange extending from the surface of said base opposite said cavity. In a further embodiment, said cavity is framed by a flat surface and optionally the flat surface is inclined from its exterior periphery towards said cavity. In a further embodiment, a surface area of said cavity is greater than a surface area of said flat surface. In a further embodiment to any of the other embodiments of this paragraph, said flat surface includes a beveled or rounded interior edge around said cavity and/or a beveled or rounded exterior edge. In a further embodiment to any of the other embodiments of this paragraph, said base having a beveled or rounded interior edge around said cavity and/or a beveled or rounded top exterior edge. In a further embodiment to any of the other embodiments of this paragraph, said cavity is a variating surface with a non-uniform level of curvature over its surface or said cavity includes a concave surface. In a further embodiment to any of the other embodiments of this paragraph, said optional plug is a Morse taper central plug. In a further embodiment to any of the other embodiments of this paragraph, said modular component includes or is made of one or more metals, and further includes Cobalt-Chromium or TiN coated Ti5Al4V.

In at least one embodiment, a modular shoulder prosthesis system includes a glenoid baseplate, a humeral baseplate or humeral base, any of the glenoid modular embodiments for ta-TSR described above, and any of the humeral modular components for ta-TSR described above.

In any of the modular component embodiments above, the component of the humeral modular component that contacts the glenoid modular component includes or is made of polyethylene and the component of the glenoid modular component that contacts the humeral modular component includes or is made of metal.

### IV. BRIEF DESCRIPTION OF THE DRAWINGS

Any cross-hatching or shading present in the figures is not intended to identify or limit the type of material present for the element shown in cross-section. In figures that include multiple elements shown in cross-section, the cross-hatching will be different directions for the different elements. Some figures include one or more components shown transparently and/or in phantom.

Any reference to top, bottom, and other orientations are based on if the component was sitting on a table unless otherwise noted, for example when describing orientation after the component is implanted into a patient.

FIGS. 1A-1C illustrate a top view, a top perspective view, and a side view, respectively, of an example of a baseplate that includes an optional fixation attachment hole passing through its base according to at least one embodiment of the invention.

FIGS. 2A-2D illustrate a top perspective view, a side view and a top view, respectively, of a glenoid modular component according to at least one embodiment of the invention.

FIGS. 3A-3K illustrate a humeral modular component. FIG. 3A illustrates an exploded, cross-sectional view of a humeral modular component and an example baseplate. FIGS. 3B and 3C illustrate a top perspective view and side view of a shell. FIGS. 3D-3G illustrate a top view, a perspective view, a side view, and a bottom view of an inner head. FIGS. 3H-3K illustrate an assembled humeral head with a bottom perspective view, a side view, a side phantom view, and a bottom view.

FIGS. 4A and 4B illustrate an example of how the modular components of FIGS. 2A-3K may interact with each other according to at least one embodiment of the invention.

FIGS. 5A-5E illustrate a side view, a bottom perspective view, a second side view, a bottom view, and a transparent view, respectively, of a glenosphere according to at least one embodiment of the invention.

FIGS. 6A-6H illustrate a humeral modular component that is a humeral cup according to at least one embodiment of the invention. FIGS. 6A-6C illustrate a top perspective view, a side view, and a top view, respectively, of the humeral cup base. FIGS. 6D-6G illustrate a top view, a top perspective view, a side view and a bottom view, respectively, of the humeral cup insert. FIG. 6H illustrates the humeral cup attached to a baseplate.

FIG. 7 illustrate how the modular components of FIGS. 5A-6G may interact with each other according to at least one embodiment of the invention.

### V. DETAILED DESCRIPTION OF THE DRAWINGS

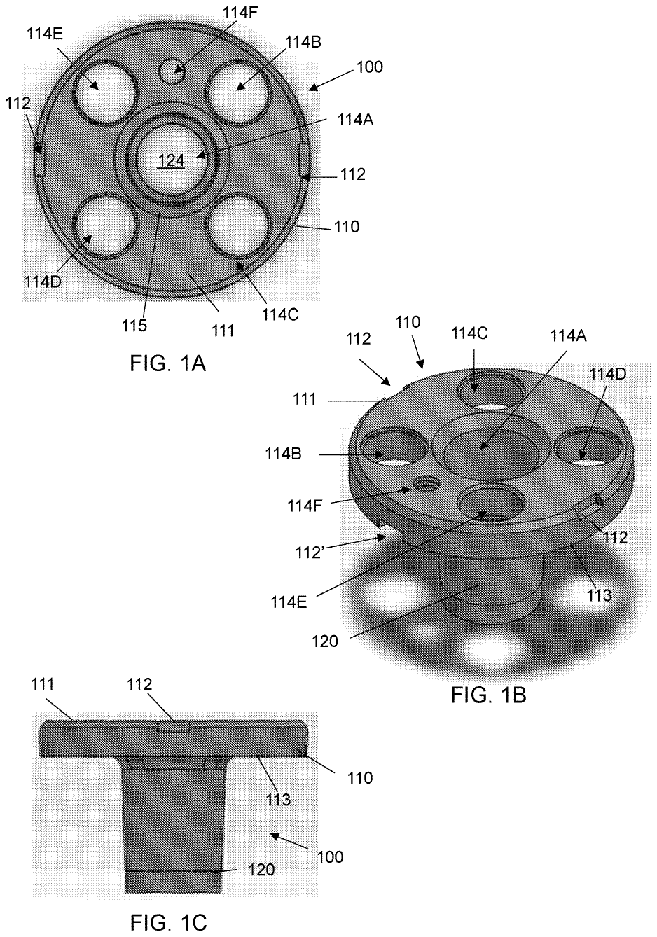

The invention in at least one embodiment includes one or more modular components, for example glenoid and/or humeral modular components, for use with baseplates disclosed in U.S. Pat. No. 10,583,012, issued on Mar. 10, 2020, and PCT Application No. PCT/US21/20492, published as WO 2021/178418 A1 on Sep. 10, 2021 and filed on Mar. 2, 2021, which are hereby both incorporated by reference for their teachings regarding different baseplates and humeral stems, which collectively will be referred to as “baseplates” in this disclosure unless otherwise indicated. FIGS. 1A-1C illustrate another example of a baseplate that includes a fixation attachment hole.

In at least one embodiment, the modular component includes an outer peripheral flange that overlaps with the baseplate that in at least one embodiment include a pair of attachment points extending in from opposed external peripheral sides to engage notches in the baseplate.

The baseplate has a mounting surface for engagement of a modular component. The mounting surface refers to the substantially planar surface of the baseplate opposite the glenoid (or humerus). The attachment in at least one embodiment between the modular component and the baseplate is through, for example, a Morse taper, which may be a dual threaded Morse taper that is axially located with reference to the baseplate. In an alternative embodiment with or without the Morse taper, a torque limiting fastener, such as a screw or a bolt, is used to further secure the glenoid modular component to the baseplate by engaging the baseplate and/or the central attachment mechanism anchored in the patient's bone. In a further embodiment, the attachment is facilitated with a threaded connection where the modular component is screwed into the baseplate. In a further embodiment, the baseplate includes a screw fixation hole or chamber. In a further embodiment, one or more of these attachment embodiments may be used in combination.

In at least one embodiment the modular shoulder replacement system further includes a humeral stem (as an alternative to a baseplate) for attachment to a patient's humeral bone. The humeral stem having a receiving socket for insertion of a post (or other connection piece) from a modular humeral head for a ta-TSR or a modular humeral cup for a r-TSR. In at least one embodiment, the humeral modular component is attached to the humeral stem using, for example, a Morse taper, which may be a dual threaded Morse taper. In an alternative embodiment, the system includes a mounting base that is attached to the humeral stem component on to which the modular humeral head or modular humeral cup is attached. In an alternative embodiment with or without the Morse taper, a torque limiting fastener, such as a screw or a bolt, is used to further secure the humeral modular component to the humeral stem by engaging the receiving socket. In a further embodiment, the attachment is facilitated with a threaded connection where the humeral modular component is screwed into the humeral stem. In a further embodiment, the attachment is facilitated with a threaded connection where the modular component is screwed into the baseplate. In a further embodiment, the baseplate includes a screw fixation hole or chamber. In a further embodiment, one or more of these attachment embodiments may be used in combination.

In an alternative embodiment, a baseplate used for the glenoid is adapted for use instead with the humerus in place of the humeral stem. In at least one embodiment, the glenoid baseplate (e.g., a first baseplate) and the humeral baseplate (e.g., a second baseplate) are identical. In at least one alternative embodiment, the humeral baseplate is larger than the glenoid baseplate. In a further embodiment, the humeral baseplate has a larger diameter (e.g., for the base and/or the stem), height, and/or thickness than the glenoid baseplate. Examples of diameters for the baseplates include about 25 mm, 27 mm, 29 mm, 32 mm, 33 mm, 37 mm, 41 mm, 42 mm, a range between 25 mm and 41 mm, a range of 25 mm to 33 mm, a range of 25 mm to 30 mm or a range of 25 mm to 27 mm where the measurements in this disclosure also include approximations given manufacturing tolerances and the ranges in a further embodiment include their respective endpoints. In at least one embodiment, the glenoid baseplate has a diameter of 27 mm while the humeral baseplate has a larger diameter although the same size diameter may be used on both the glenoid and humeral sides. Examples of baseplate sizes include 39 mm, 41 mm, 43 mm, 46 mm, 49 mm, 52 mm, and 55 mm, and in at least one further embodiment, these sizes are used for a humeral baseplate. Examples of humeral head thicknesses include 14 mm, 17 mm, and 20 mm. In at least one embodiment, the humeral head has an offset center in which there is a neutral position.

The baseplate and the humeral stem (or a second baseplate) are designed to remain in place while switching the modular components to switch from a ta-TSR to a r-TSR. However, there may be times the baseplate needs to be extracted using one or more tools that engage the plurality of attachment points.

FIGS. 1A-1C illustrate an example of a baseplate 100 having a plurality of mounting holes 114A-114E and an optional fixation attachment hole or chamber 114F (fixation attachment hole will be used herein to cover fixation attachment chamber unless explicitly stated otherwise) that can be used to attach the modular component, for example a glenosphere, to the baseplate 100.

The illustrated baseplate 100 includes a base 110 and a stem 120 extending from the base 110. Although the base 110 is illustrated as being circular, the base 110 may be elliptical, oval, or other suitable shapes; in such an embodiment, the modular component may be shaped to match. Examples of the thickness of the base 110 include between 5 mm and 15 mm (with or without the end points), 5 mm, 7 mm, 10 mm, 12.5 mm, and 15 mm. In at least one embodiment, the baseplate 100 is made from an ingrowth trabecular metal over a metal core of, for example, steel, titanium, titanium alloy (e.g., Ti6Al4V), or a combination of two or more of these. In an alternative embodiment, the ingrowth trabecular metal is omitted. The ingrowth trabecular metal facilitates bone ingrowth into the baseplate 100, for example to increase the strength of the connection between the bone and the baseplate 100 and the respective interface shear strength over time.

Although the stem 120 is illustrated as a cylinder with a slight taper, in at least one embodiment, the stem 120 has tapered sides to match the modular component plugs. In a further embodiment, the stem 120 is substantially cylindrical.

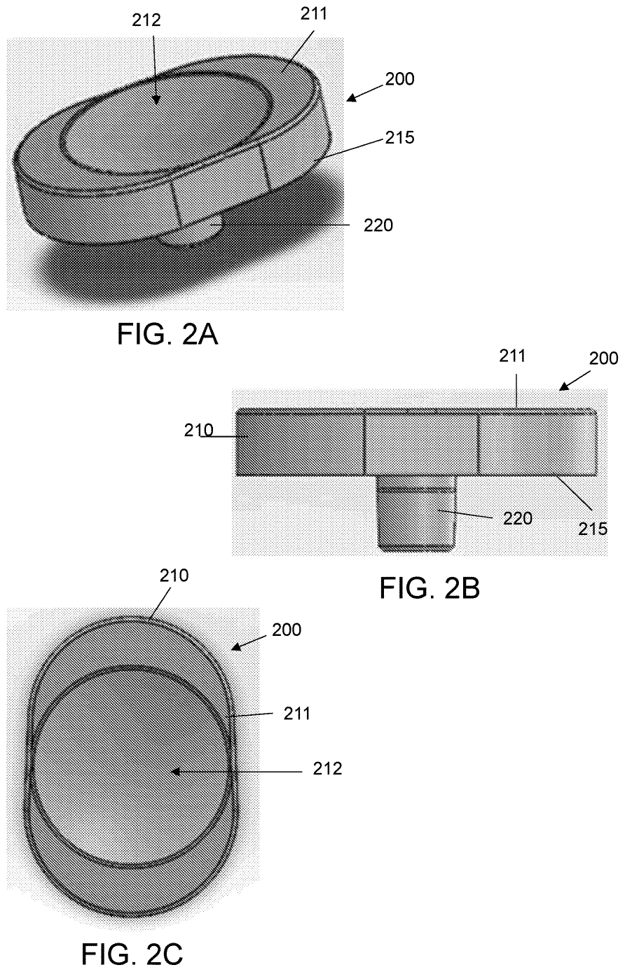

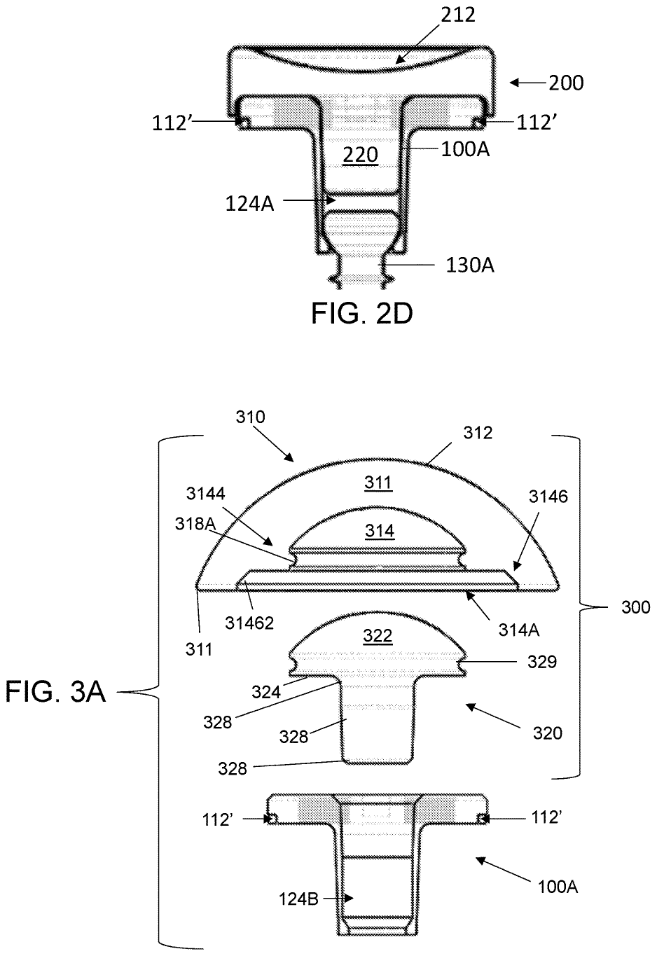

The base 110 includes a mounting surface 111 on a side opposite of the side 113 from which the stem 120 extends. In at least one embodiment, the mounting surface 111 is substantially planar. The plane defined by the mounting surface 111 is approximately parallel to the resection plane after implantation assuming there is no use of a wedge. In an alternative embodiment, the plane defined by the mounting surface 111 is at an angle to the resection plane after implantation. The mounting surface 111 in at least one embodiment will be a sufficient height above the glenoid resection plane to allow access to notches 112, 112 (extending down from the mounting surface 111) or attachment points 112′, 112′ (extending up from the opposite side 113 and both illustrated, for example, in FIG. 2D). In at least one embodiment, the notches and/or the attachment points are omitted.

The illustrated base 110 includes a pair of attachment points, which are illustrated as notches 112′, 112′. See, e.g., FIG. 2D. The notches extend up from outer circumferential sides of the bottom surface 113 from which the stem 120 extends. The notches 112′ are present on the outer circumferential sides of the base 110. The notches 112′, 112′ are accessible from the exterior of the baseplate 100. The notches 112′, 112′ have sufficient width and depth to engage with an implanting/extracting instrument. Although two attachment points are discussed, additional attachment points could be added to the base 110.

The illustrated base 110 includes a pair of opposed leverage notches 112, 112 extending down from outer circumferential sides of the mounting surface 111, for example on the anterior and posterior central exterior edge (or side) of the base 110, which in at least one embodiment provides better access to the notches 112, 112 for removal of the modular component. The notches 112, 112 are configured to be accessible from the mounting surface 111. In at least one embodiment, the notches 112, 112 provide (or are configured to have) a leverage point to facilitate separation of the mounted modular component from the baseplate 100 when the baseplate 100 is implanted. The notches 112, 112 have sufficient width and depth to receive an instrument in which to pry the mounted modular component from the baseplate 100. Although two notches 112, 112 are illustrated, additional notches could be added to the base 110.

In at least one embodiment where leverage notches are present, the leverage notches and the attachment points are aligned with each other, while in an alternative embodiment the leverage notches and the attachment points are offset from each other as illustrated in FIG. 1B.

The illustrated base 110 includes five mounting holes 114A-114E with an axially centered hole 114A and four evenly spaced perimeter holes 114B-114E around the mounting surface 111. The holes 114A-114E are illustrated as having a shoulder 115 on which a screwhead, which is an example of an attachment mechanism, will make contact after insertion into the baseplate 100. Although five holes are illustrated, the number of holes could be reduced, including omission of the perimeter holes, or increased. In at least one embodiment, the central hole 114A defines a chamber 124 for receiving a modular component plug. The chamber 124 may optionally include a beveled opening. In at least one embodiment, the holes 114B-114E are offset from the notches 112, 112. For example, hole 114B might be at approximately 1 or 2 o'clock while hole 114E might be at approximately 10 or 11 o'clock if the notches 112, 112 are at 3 and 9 o'clock, respectively. An alternative embodiment does not offset the openings 114C and 114 E are not offset with notches 112, 112, 112′, 112′.

In at least one embodiment, one or more attachment mechanisms include variable angle locking screws that are used to attach the baseplate 100 to the patient's bone. Examples of screw diameters include 4.0 mot to 4.5 mm to 5.0 mm and in a further example including the end points of that range, and more particularly 5.0 mm. Although there are five holes illustrated, during a particular procedure, all five holes may not be utilized. In at least one embodiment, the flexibility in which holes 114A-114E to use and the variable angle locking screws provides flexibility to the orthopedic surgeon in securing the baseplate 100 to the patient's bone. Examples of locking screw angles includes between 20 degrees and 30 degrees (with or without the end points) or perpendicular to the base 110. Additional examples of attachment mechanisms include screws, variable angle locking screws, and an impactable central cylinder.

In at least one embodiment, the central axial opening 114A passes from the base 110 into and through the stem 120 to allow for the top of the locking screw 130A to be deeper into the baseplate 100 and to provide the chamber 124A for receiving a plug, e.g., the Morse taper, of the modular component being mounted onto the baseplate 100A as illustrated, for example, in FIGS. 2D, 5E, and 6H.

FIG. 1C illustrates an example of how the outer peripheral edge of the mounting surface 111 may be tapered to reduce or prevent edge stresses. FIG. 1C also illustrates the presence of a curved transition from the bottom surface 113 to the stem 120.

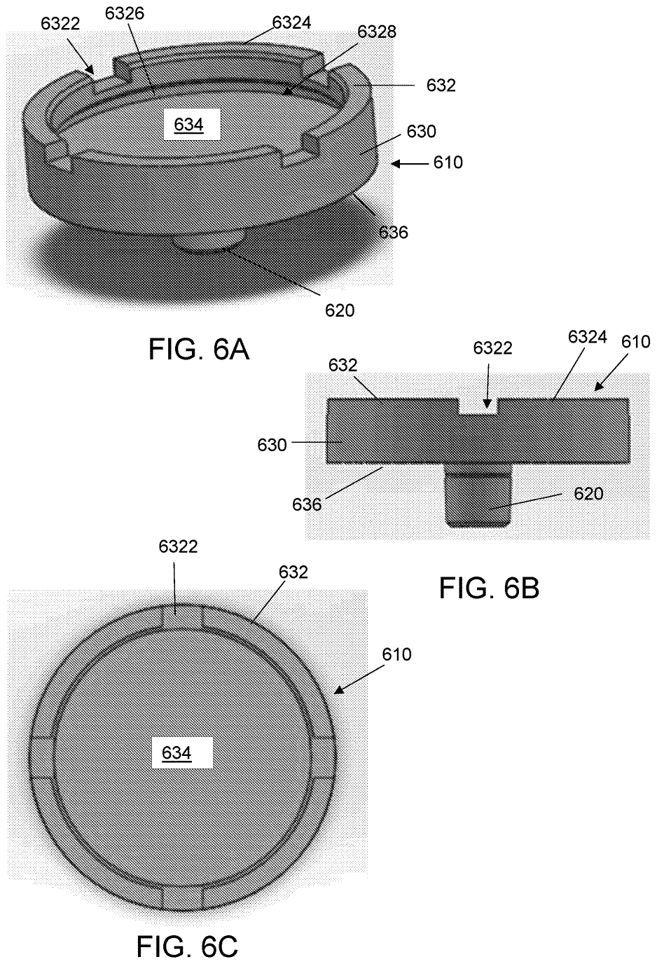

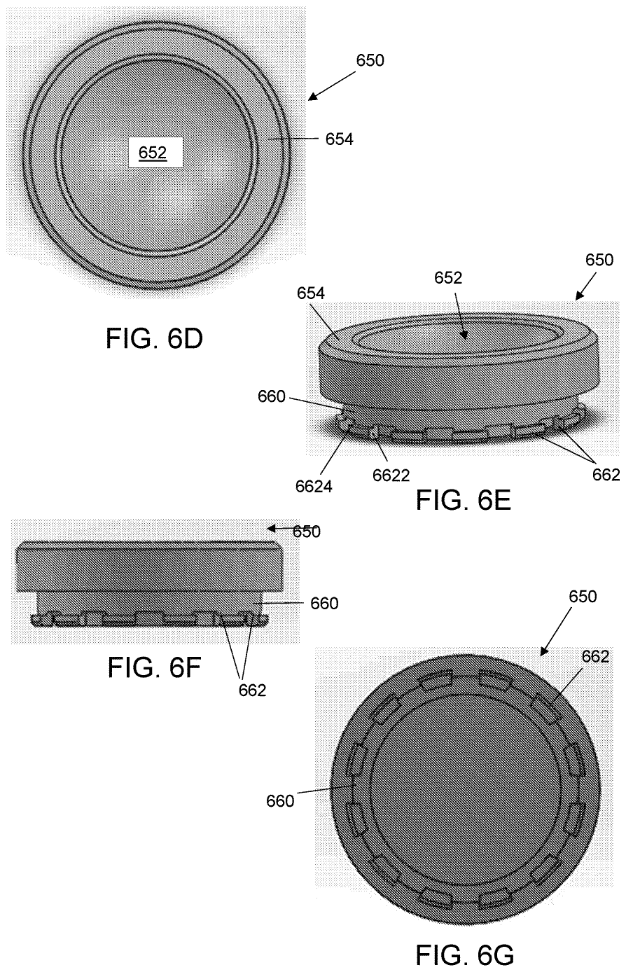

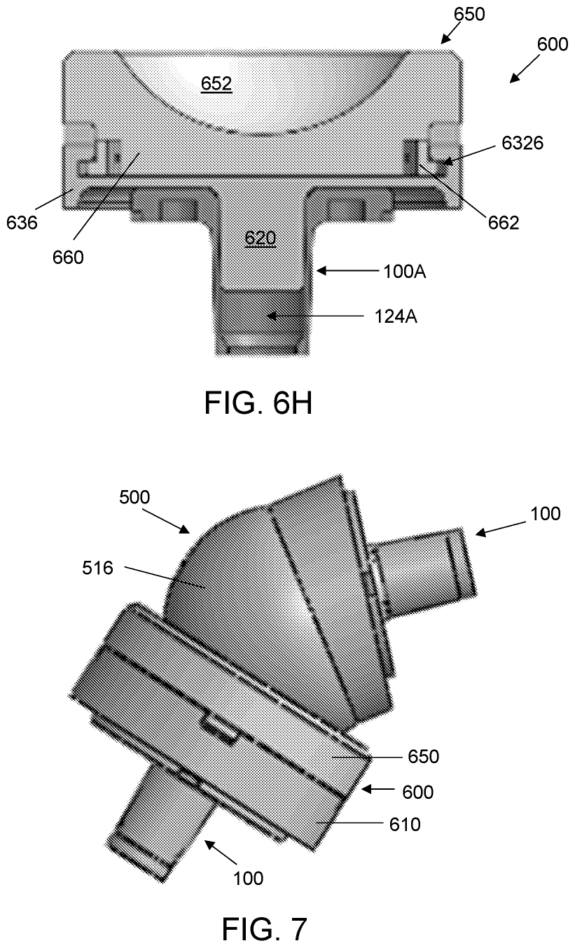

In at least one embodiment to the previous baseplate embodiments, the fixation attachment hole 114F provides a further connection point between the baseplate 100 and the modular component that locks the orientation between the baseplate 100 and the modular component as illustrated, for example, in FIG. 6E. As illustrated in FIG. 1B, the fixation attachment hole 114F is a threaded hole that is configured to engage a fastening screw 131 extending from the modular component. In use, the fastening screw may pass through the baseplate and anchor into the bone or stop within the fixation attachment hole 114F. In an alternative embodiment where the fastening screw 131 does not pass through the baseplate 110, then the fixation attachment hole 114F may instead be a chamber that receives the fastening screw. In an alternative embodiment, the fixation attachment hole 114F is not threaded and the fastening screw passes therethrough and is anchored into the patient's bone. In an alternative embodiment to the embodiments in this paragraph, the fastening screw is replaced with any of the anchoring mechanisms previously discussed.

Based on this disclosure, a person having ordinary skill in the art should appreciate that the modular components described herein may be used with different baseplates like those described in U.S. Pat. No. 10,583,012, issued on Mar. 10, 2020, and PCT Application No. PCT/US21/20492, published as WO 2021/178418 A1 on Sep. 10, 2021 and filed on Mar. 2, 2021. Based on this disclosure, a person having ordinary skill in the art should appreciate that the other baseplates in this disclosure along with the reference to previously described baseplates in these prior applications may be modified to include a fixation attachment hole. Although similar structures present in the other baseplates illustrated in this disclosure, a person having ordinary skill in the art should appreciate that structures similar to those shown in FIGS. 1A-1C will be present in these other baseplates.

FIGS. 2A-2D illustrate an example of a glenoid modular component for use in ta-TSR. The illustrated glenoid modular component 200 includes a concave cavity 212 in the engagement surface 211 on a base 210 for receiving a prosthetic ball, spherical object, or another convex shaped interface and a plug 220 extending from a bottom surface 215 opposed to the concave cavity 212. One of ordinary skill in the art should appreciate based on this disclosure, the concave surface of the cavity 212 can take a variety of shapes without departing from the modularity of the glenoid modular component 200. An example of a different shape is a variating surface with a non-uniform level of curvature over the concave surface. The plug 220 is configured to be inserted and frictionally engage the chamber 124 in the baseplate 100. In at least one embodiment, the plug 220 is a Morse taper central plug.

The glenoid modular component 200 is illustrated as being elliptical with a substantially spherical concave cavity 212 centrally located. As illustrated, the concave cavity 212 may be framed with a flat surface, which in an alternative embodiment may be a partially funnel shape towards the concave cavity 212. In an alternative embodiment, the concave cavity 212 covers more of the surface area of the engagement surface 211 than the flat surface area. Advantages to this shape include providing greater conformity and stability of the prosthetic components.

As illustrated, the engagement surface 211 includes a beveled or rounded outer edge and a beveled or rounded edge around the concave cavity 212. The beveled edge(s) reduces and/or prevents edge stress loading. In a further or alternative embodiment, the bottom surface 215 may also include a beveled or rounded outer peripheral edge.

In at least one embodiment as illustrated in FIG. 2D, the modular component 200 fits over the baseplate 100A with a peripheral flange 230 extending down from the bottom surface 215 of the modular component. FIG. 2D also illustrates the use of a screw fixation central post 130A to secure the baseplate 100A to the glenoid.

In at least one embodiment, the glenoid modular component 200 is manufactured from Cobalt-Chromium (Co—Cr) or TiN coated Ti6Al4V to improve the life expectancy for the implant. In an alternative embodiment, the glenoid modular component 200 includes or is made of metal.

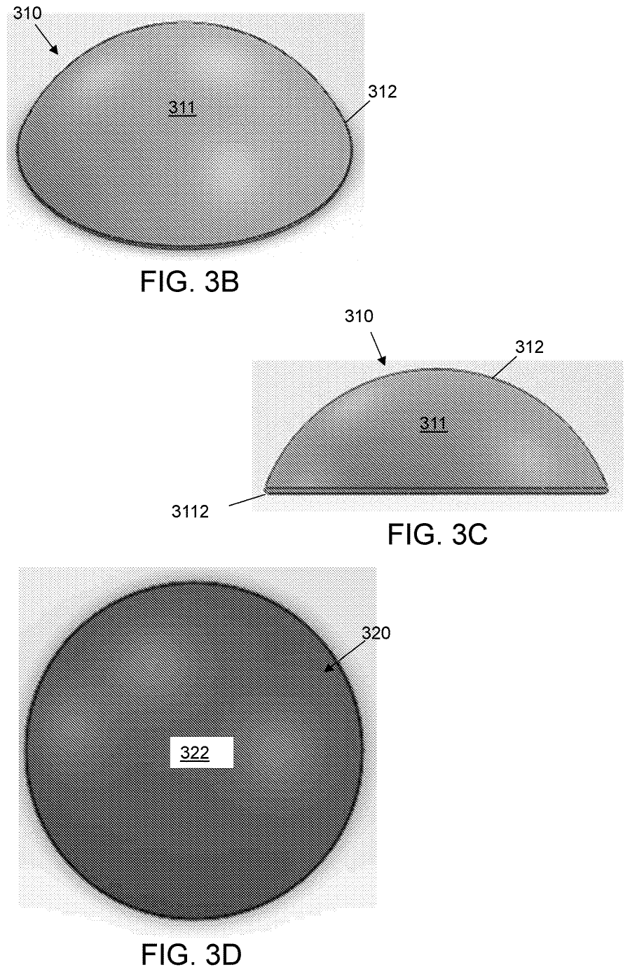

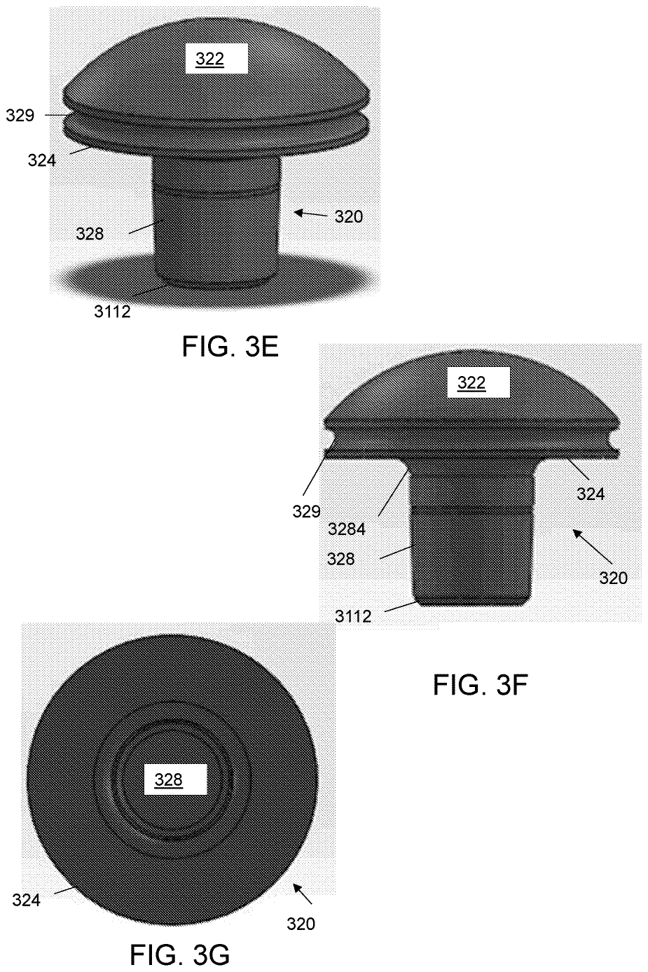

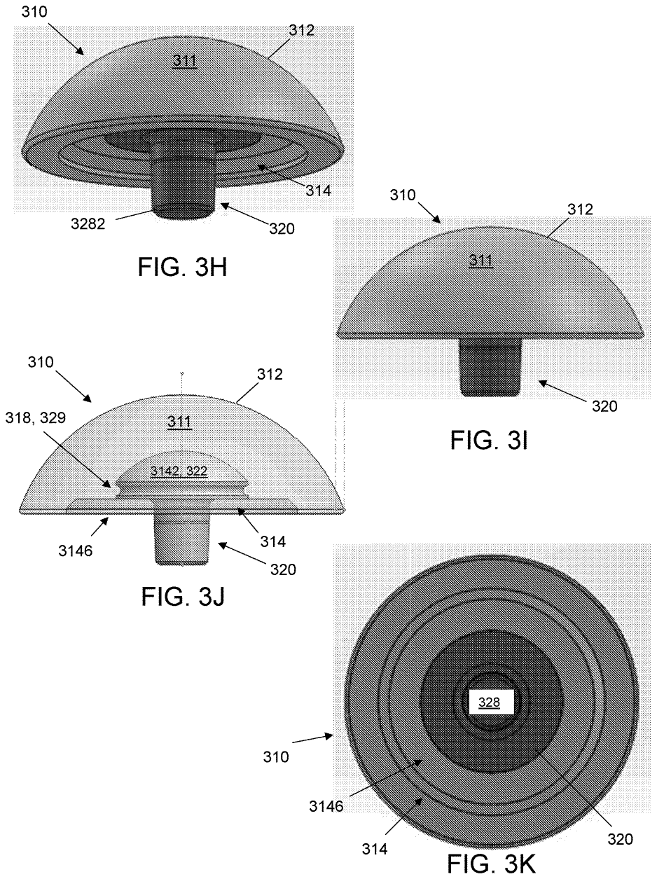

FIG. 3A illustrates an exploded, cross-sectional view of the humeral modular component 300 that includes a shell (or outer poly shell) 310 and an inner head (or humeral head base) 320.

As illustrated in FIGS. 3A-3C, the shell 310 includes a body 311 having a generally hemispherical (or convex) surface 312 and defining an interior cavity 314 into which the inner head 320 is inserted. The exterior surface 312 is configured to interact with a concave surface of a glenoid modular component like that discussed in the previously identified applications, PCT Application No. PCT/US2022/026940 and in connection with FIGS. 2A-2D supra. The interior cavity 314 includes a concave section 3142 into which the dome 322 of the inner head 320 fits with the outer peripheral edge including an engagement channel 329 that engages an engagement section 3144 with a ring protrusion 318 of the interior cavity 314 as illustrated in FIGS. 3A and 3J. The engagement surfaces 318, 329 are complementary and in an alternative embodiment are reversed to each other. The engagement channel of the inner head 320 includes a channel 329 with shoulders on either side into which the ring protrusion 318 fits to secure the two pieces together. In at least one embodiment, the bottom section 3146 of the interior cavity 314 is large enough to fit over the baseplate in a manner similar to the previously discussed flange. In a further optional embodiment, the bottom section 3146 includes an inwardly tapered wall 31462 as illustrated in FIGS. 3A, 3H, 3J, and 3K, which tapered wall provides a substantially even thickness for the outer shell in around that section of the cavity. In a further embodiment, the taper wall 31642 may further assist with insertion of the inner head 320 with its dome 322 and over the baseplate 100A, which includes a variant of the central chamber 124B, particularly in the situation where the inner head 320 has been implanted and the outer shell 310 is being replaced. In at least one embodiment, the bottom section 3146 has a diameter for its opening that is just slightly larger than the baseplate diameter. In an alternative embodiment, the bottom section 3146 is omitted from the outer shell 310 leaving the bottom of the outer shell to abut or rest substantially against the baseplate.

FIGS. 3A-3C also illustrate an alternative embodiment for the body 311 that includes a beveled outer edge 3112 around the bottom of the body 311. The beveled edge 3112 reduces (or eliminates) loading pressure along that edge that could lead to fatigue and/or failure resulting in deterioration of the outer shell and possibly breaking off pieces from the shell edge into the patient's body. In at least one embodiment, the beveled or arcuate edge is added to an outer shell that may have a different internal structure than that illustrated in FIGS. 3A and 3J. In alternative embodiments, one or more of the beveled edges are rounded.

The illustrated inner head 320 includes a dome 322, a base 324, and a plug 328 for insertion into a receiving cavity of the baseplate embedded into the patient's humeral bone. In at least one embodiment as illustrated in FIGS. 3A, 3E, and 3F, the plug 328 may include a beveled or rounded surface where it extends from the base 324. An advantage of using an inner head is that it can be used with different sized shells to provide connection between the shell and the baseplate, and also allows for the replacement of the shell if it were to wear out while leaving the inner head in place. The dome 322 will further assist with mounting of the shell 310 on the inner head 320, particularly when the inner head 320 is already implanted in the patient. When the inner head base 324 has a smaller diameter than the mounting surface of the baseplate 100A, the mounting surface can provide a stop for the shell 310.

FIGS. 3A, 3E, and 3F illustrate the interface to engage the attached shell. The illustrated interface includes a channel 329 that is formed on the peripheral outside of the base 324. Above and below the channel 329 are shoulders. In at least one embodiment, the channel and shoulders form a groove. The channel 329 although illustrated as having a concave surface when viewed in a cross-section, see, e.g., FIG. 3A, other cross-sections are possible while having the channel 329 and the protrusion 318 of the shell having complementary shapes to each other. The illustrated shape allows for the shell 310 to rotate relative to the inner head 320. In an embodiment where there is a small gap between the channel 329 and the protrusion 318, then the shell 310 may move in the radial direction relative to the inner head 320.

FIGS. 3A and 3E-3K illustrate the presence of a Morse taper for the plug 328 that includes a beveled tip 3282 and also a shaped interface 3284 between the post 328 and the base 324. In at least one embodiment, the shaped interface 3284 is beveled, while in an alternative embodiment it is arcuate shaped. The baseplate 100A illustrated in FIG. 3A includes a complementary cavity 124A that includes a tapered surface to match the Morse taper.

In an alternative embodiment, the shell is a dual mobility shell.

In at least one embodiment, the largest head diameter at a minimum thickness is 58 mm×18 mm. Additional examples of head diameters includes 38 mm to 52 mm with or without end points with additional sizes including 38 mm, 40 mm, 42 mm, 44 mm, 46 mm, 48 mm, 50 mm, and/or 52 mm. Additional examples of thicknesses include 15 mm, 18 mm, 21 mm, 15 mm to 18 mm, and 18 mm to 21 mm with the ranges with or without end points.

Examples of material for the shell include UHMWPE-highly crosslinked, a Vitamin E doped polyethylene, and Vitamin E HDPE, along with any other alterations of modern polyethylene compositions. Examples of material for the inner head include Ti6Al4V, a titanium-aluminum alloy, and cobalt chrome among other metals.

In a further embodiment to any of the above humeral modular component embodiments, the outer shell is bonded to the inner head. In a further embodiment, the shell is molded around the inner head, which in a further alternative embodiment would allow for the engagement channel and section between the shell and the inner head to be omitted.

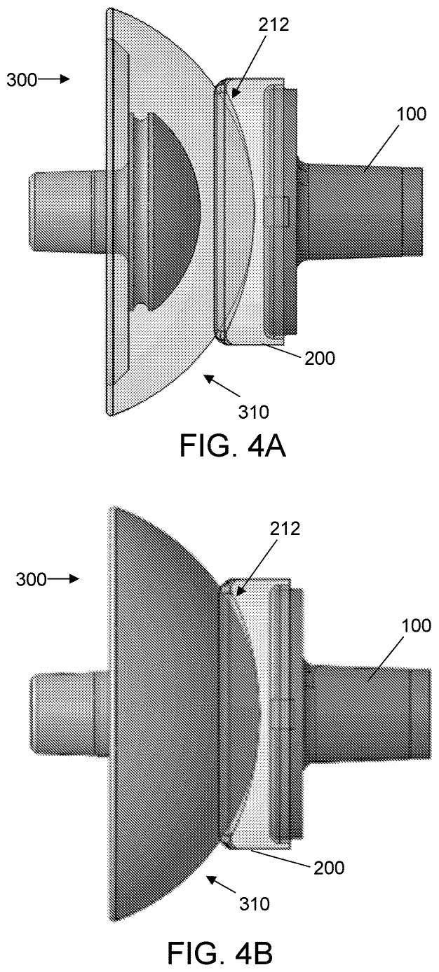

FIGS. 4A and 4B illustrate how the TSR modular components of FIGS. 2A-3K have complementary shapes and the glenoid modular component attaches to a baseplate 100. Both of these figures illustrate how the shell 310 fits into the area defined by the concave cavity 212 while allowing the humeral modular component 300 to rotate and/or move backward, forward, and sideways relative to the glenoid modular component 200.

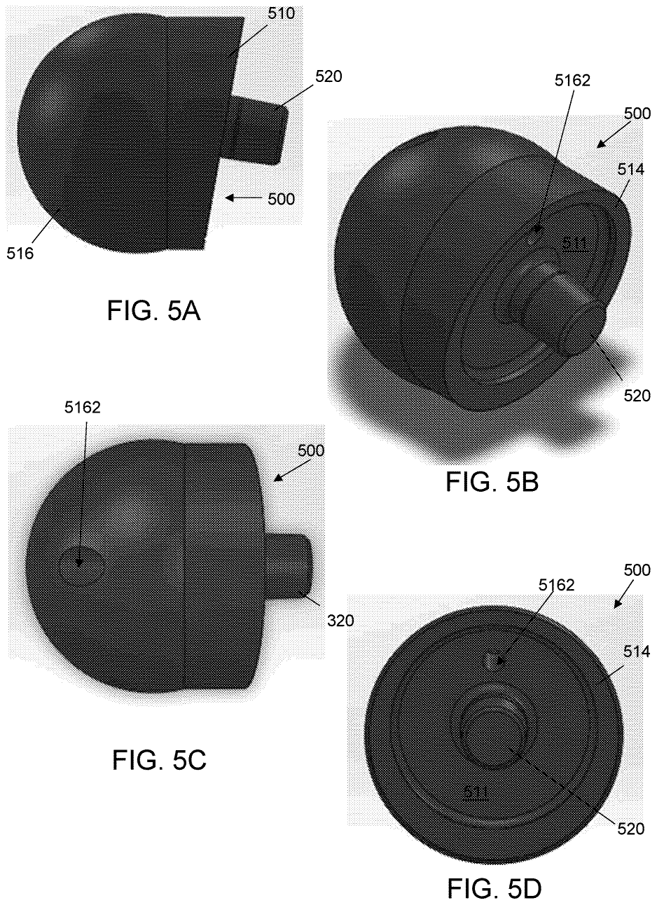

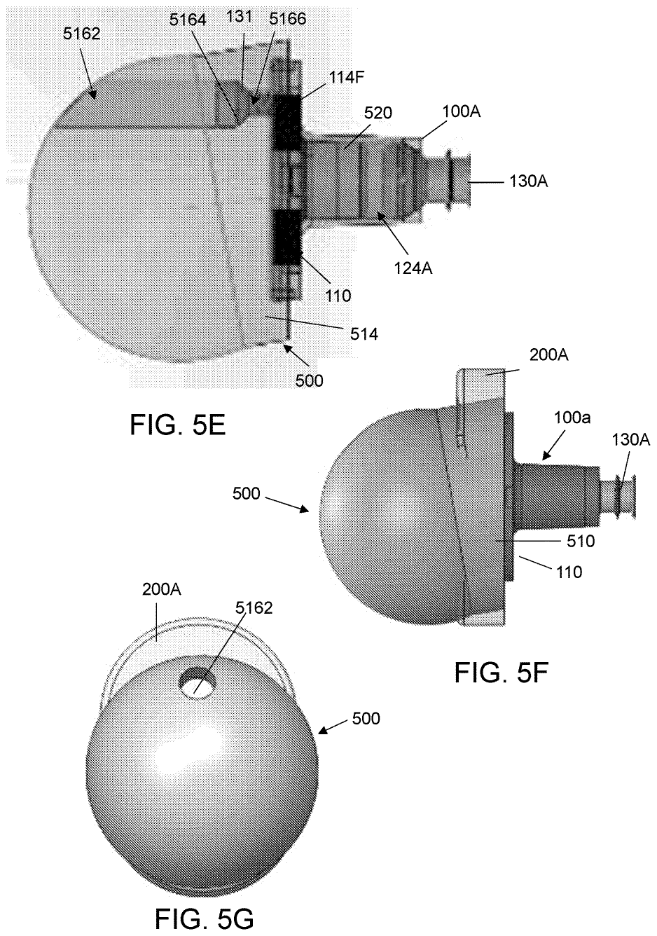

FIGS. 5A-5E illustrate a glenosphere having an attachment passage to affix it to the fixation attachment hole 114F of the baseplate 100 illustrated in FIGS. 1A-1C.

The illustrated glenosphere component 500 includes a glenosphere 516 extending from a base 510 and a plug 520 extending from a baseplate engagement face 511 of the base 510. In at least one embodiment, the plug 520 is similar to the plug 220 discussed above in connection with the glenoid modular component 200 including any further or alternative embodiments.

In at least one embodiment, there is a 7-10 degree inferior tilt or approximately 10 degree inferior tilt added to the glenosphere that allows for improved glenosphere positioning for particular patient situations. In a further embodiment, the inferior tilt is approximately 10 degrees. “Approximately” is being used here to accommodate manufacturing tolerances of 2 or fewer degrees. The inferior tilt is measured from a reference axis that extends through the base 510 and is perpendicular to the baseplate engagement face 511 of the base 510, which is the face that abuts against the baseplate. When the glenosphere 516 is implanted onto a baseplate, the reference axis will be substantially parallel to the ground and horizontal using this orientation or at a slight angle to reflect the angle of recission of the glenoid surface. In at least one embodiment, the reference axis is centered with the plug 520. There are a major axis and a minor axis that intersect at the reference axis. The minor axis is from the anterior side to the posterior side, while the major axis is perpendicular to the minor axis similar to the definition of a major axis for an ellipse. In at least one embodiment, the plug 520 will be centered as to the minor axis, but off-centered along the major axis when viewed from the anterior side or the posterior side, and in a further embodiment will be on the superior side of center along the major axis.

In a further embodiment, the inferior mass of the glenosphere 516 protrudes from the base 510 at an angle by approximately 7 to approximately 10 degrees or approximately 10 degrees inferiorly from the reference axis. The presence of an inferior tilt in the glenosphere 516 avoids the need to remove the baseplate and to remove additional glenoid bone to provide a suitable angle for engagement of a non-tilted glenosphere for use with a humeral cup 600, for example like that illustrated in FIGS. 6A-6G. In at least one embodiment, the majority of the mass of the glenosphere 516 is on the inferior side of the plug 520 thus providing a spherical area aligned with the humeral cup 600 and providing a closer approximation to the natural movement of the humerus relative to the glenoid. See, e.g., FIG. 7. In at least one embodiment, when the glenosphere 316 is viewed from the side after implanting, the majority of the mass is below a horizontal axis passing through the axial center of the baseplate. See, e.g., FIG. 5E.

In at least one embodiment, the center of rotation (COR) for the glenosphere 516 will have a 10 mm lateralization of the COR with a diameter of 52 mm. In a further embodiment, the “minus” sizes in 4 mm increments will allow for a 6 mm lateralization to diminish glenoid bone-prosthesis surface shear stresses. Examples of this concept include glenosphere diameters of 52 mm, 56 mm, and 40 mm with minus options to keep lateralization to 10 mm or 6 mm such that 52 mm neutral and 52 mm “−4”, 56 mm (is already a “−4”) and a 56 mm “−4” (which results in a “−8”), 40 mm (by definition is a “−8”) and a 44 mm “−4” (which results in a “−12”).

The shape of the glenosphere 516 may vary from that illustrated and the shape illustrated in these figures are not intended to limit the exact shape as these are illustrative figures.

As illustrated in FIG. 5E, a locking screw 131 may be inserted through the attachment passage 5162 to engage the fixation attachment hole 114F. The passage 5162 illustrated in FIG. 5E includes a shoulder (or beveled section) 5164 on which the fastening screw 131 may contact to hold the glenosphere 516 in place relative to the baseplate 100A. In a further embodiment, the passage 5162 below the shoulder 5164 may be threaded section 5166 to further secure the fastening screw 131 to the glenosphere 516. The attachment passage 5162 is located along the major axis near the superior peripheral edge as illustrated, for example, in FIGS. 5D, 5E, and 5G. The locking screw 131 can provide additional anchoring of the glenosphere to the baseplate to counteract the mass distribution relative to the baseplate. In an alternative and/or further embodiment for the other modular components, the attachment passage may be added to them to provide an additional anchoring point for those modular components.

FIGS. 5B and 5D illustrate how the glenoid modular component may include a flange 514 extending from the bottom of the base 510. The flange 514 extends down from bottom surface and the outer circumferential edge of the base 510. The flange 514 is configured to fit over and down at least a portion of the peripheral external sides of the baseplate 100A. In this embodiment, the base 510 has a wider diameter than the base 110 of the baseplate 100. FIG. 5D illustrates how the thickness of the flange 514 varies around its circumference to facilitate a smooth exterior surface of the offset glenosphere 516. The thicker flange along the inferior side assists with stability for the inferior offset of the glenosphere 516 and resulting 10 degree inferior tilt to square the modular component.

FIGS. 5E and 5F illustrate another example of the use of a screw fixation central post 130A to secure the baseplate 100A to the patient's glenoid. As illustrated in FIG. 5E, there may be a space between the bottom of the plug 520 and the top of the screw fixation central post 130A within the chamber 124A.

FIGS. 5F and 5G illustrate how in at least one embodiment, the glenosphere modular component 500 may sit lower after being implanted than a glenoid component 200A (partially superimposed in these figures) in a ta-TSR and thus the glenosphere 516 may sit eccentrically lower on the implanted baseplate 100A as illustrated in these figures. Advantages to this lower sitting include 1) inferior notching migration from the glenosphere sitting at the inferior edge of the glenoid and 2) obviates revision by avoiding the need to remove the baseplate and/or harvesting local autograft, which both shorten surgical times and improves patient recoveries. This configuration is designed to address inferior placement concerns with built-in 10° of inferior angulation and 3 mm offset creating an equivalent starting hole (4 mm inferior) to the original baseplate center.

In at least one alternative embodiment of the illustrated glenosphere 516 and discussed glenospheres, the affixation hole 5162 is omitted.

In at least one further embodiment to the other glenosphere embodiments, the glenosphere 316 is made from Co—Cr or, alternatively, other metal(s).

FIGS. 6A-6G together illustrate a humeral cup that includes a humeral cup base 610 (FIGS. 6A-6C) for attachment to a baseplate and a humeral cup shell 650 (or an insert) (FIGS. 6D-6G) for attachment to the humeral cup base 610.

In at least one embodiment, the humeral cup base 610 for a r-TSR is made from Co—Cr or, alternatively, other metal(s), for example when the glenosphere component 500 has a high-density polyethylene cover, layer, and/or coating on it. The illustrated humeral cup base 610 includes a body 630 from which a wall 632, which may be cylindrical, extends up and an optional flange 636, which may be cylindrical, and a plug 620 (e.g., like the plugs of the other modular components) extending downward. In at least one embodiment the cylindrical flange 636 fits over the baseplate 100A, for example as illustrated in FIG. 7. In at least one embodiment, the wall and the optional flange have corresponding and/or complementary shapes to the body.

The cylindrical wall 632, which defines a receiving cavity 634, includes optional notches 6322 that extend down from the rim 6324 of the wall 632. For example as illustrated in FIGS. 6A and 6C, the number of notches 6322 may be 4 but any number between 0-6 may be present. The notches 6322 provide a leverage point to separate an inserted humeral cup shell 650 from the base 610. In at least one embodiment and as illustrated, the cylindrical wall 632 may also include an optional groove 6326 around its interior bottom 6328 of the wall 632 for receiving tabs 662 or other protrusions on the shell 650 to facilitate securing the shell 650 to the base 610. In a further embodiment, the inner periphery edge of the wall 632 is beveled to assist with insertion of the shell 650, which may be advantageous when the shell 650 has flexibility in its bottom structure The shell 650 includes a curved cavity 652 having a curvature to it configured to allow the glenosphere 516 to move relative to it. In at least one embodiment, the curvature is a variating surface of the cavity 652 with a non-uniform level of curvature of the concave surface of the cavity 652. In a further embodiment to these embodiments, the cavity 652 is framed by a flat surface 654 that may have an optional beveled or rounded outer and/or inner periphery edge. An advantage to the presence of the beveled outer and/or inner periphery edge reduces and/or prevents edge stress loading. In an alternative embodiment to the flat surface 654, the surface may have a general funnel shape while optionally having an outer and/or inner periphery edge that is beveled or rounded. In a further embodiment to these embodiments, when the shell 650 has been worn down, then it may be removed and replaced. One approach for removing the shell 650 is prying the shell 650 from the receiving cavity 634, for example using one or more of the notches 6322 on the base 610 as an insertion point(s) for a prying tool. Another approach for removing the shell 650 is to freeze it with liquid Nitrogen to shrink it before it pops out from the receiving cavity 634. One example of the curvature of the cavity 652 is a concave surface for receiving the glenosphere 516.

The illustrated shell 650 is configured to be inserted into the receiving cavity 634 of the humeral cup base 610. The shell 650 is designed to fit within the cavity 634. The illustrated shell 650 includes a cylindrical bottom wall 660 inset from the outer edge to provide a stepped outer bottom edge (or lip) 662 configured to receive the peripheral outer wall 632 of the base 610. Extending down and/or radially out from the bottom wall 660 are tabs 662 or other protrusions that in at least one embodiment have some flexibility to engage the interior groove 6326 of the base 610. In a further embodiment, the bottom wall 660 provides additional flexibility to the shell 650 to facilitate its insertion into the base 610. In at least one embodiment, there are a plurality of tabs 662 that extend down and are spaced from each other with each tab 662 having a free end 6622 with a radially extending hook 6624 to engage the groove 6326 and optionally the wall 632. In at least one further embodiment, the wall 660 has a complementary shape to the cavity 634.

FIG. 6H illustrates how the humeral cup 600 illustrated in FIGS. 6A-6G might attach to a baseplate 100A including how the shell 650 with tabs 662 fits into the groove 6326 and engages the base 610. The cross-section is taken through a plane that intersects with two of the notches 6322 of the base 610 to illustrate the gap provided by the notches 6322. FIG. 6H also illustrates an alternative embodiment where the shell 650 does not include a cavity defined by the bottom wall 660 and the shell 650 is solid within the area defined by the wall to provide additional support and durability to the shell 650 after it is implanted.

In at least one embodiment, the shell 650 is made of or includes a coating with polyethylene or a related articulating material, or Vitamin E HDPE or, alternatively, other polyethylene fabrications.

FIG. 7 illustrates how the modular components of FIGS. 5A-6G for a r-TSA have complementary surfaces that allow them to move backward, forward, and sideways and rotate relative to each other. FIG. 7 also illustrates how the glenosphere 516 with 10 degrees of inferior inclination along with the glenoid baseplate 100A having 5 degrees of inferior inclination places the glenosphere 516 into a position that is believed to provide the greatest glenoid component stability at 15 degrees of inferior inclination. In conjunction with this arrangement, the humeral components 600, 100 are at angled at 140 degrees to provide what is believe to be the ideal angle of compression. Additionally, the greater degree of humeral and glenoid component conformity of the radius of curvature adds greater stability of the replacement shoulder joint. So together, all these factors should improve stability. Finite element analysis has shown a decrease in stress relative to other components due to this conformity.

Although particular materials have been identified for particular components and structural elements, one of ordinary skill in the art will appreciate that other materials, which are commonly present in shoulder replacement components, may be substituted without departing from the scope of the invention. In at least one embodiment, modules attached to the humeral side and the glenoid side will not both be the same material at the point of interaction.

Based on this disclosure, a person having ordinary skill in the art should appreciate that the different embodiments for each modular component may be used with different embodiments of the corresponding modular component for ta-TSR and r-TSR.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the root terms “include” and/or “have”, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means plus function elements in the claims below are intended to include any structure, or material, for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention.

As used above “substantially,” “generally,” “approximately,” and other words of degree are relative modifiers intended to indicate permissible variation from the characteristic so modified particularly when relating to manufacturing and production tolerances. It is not intended to be limited to the absolute value or characteristic which it modifies but rather possessing more of the physical or functional characteristic than its opposite, and preferably, approaching or approximating such a physical or functional characteristic.

Those skilled in the art will appreciate that various adaptations and modifications of the embodiments described above can be configured without departing from the scope and spirit of the invention. Therefore, it is to be understood that, within the scope of the appended claims, the invention may be practiced other than as specifically described herein.

Although one level of multiple dependencies is present in the claims attached to this disclosure, it should be understood that none conflicting claim recitation of dependent claims may be combined, for example, in the manner of the priority applications. Additionally, any dependent claim may be added to its base claim or any intermediary claim as an optional feature.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. A humeral modular component for use in a modular shoulder prosthesis system having a baseplate with or without notches and/or attachment points, the humeral modular component comprising:
a humeral cup base having
a receiving cavity,
a plug extending away from said base; and
a humeral cup shell configured to be removably inserted into the receiving cavity of said humeral cup base, said shell including a base having a curved cavity framed by a flat surface.

15. The humeral modular component according to claim 14, wherein said base includes a wall extending up to define the receiving cavity and said wall includes notches extending down from a top of the wall.

16. The humeral modular component according to claim 15, wherein said wall includes a beveled or rounded interior edge and/or a beveled or rounded exterior edge on its top.

17. A humeral modular component for use in a modular shoulder prosthesis system having a baseplate with or without notches and/or attachment points, the humeral modular component comprising:
a humeral cup base having
a receiving cavity,
a plug extending away from said base; and
a humeral cup shell configured to be removably inserted into the receiving cavity of said humeral cup base, said shell including a base, a curved cavity formed in said base, and a wall extending down from a surface of said base opposite said curved cavity, said wall is inset in from an outer peripheral surface of said base.

18. The humeral modular component according to claim 17, wherein said wall including a plurality of protrusions for engaging a groove around an interior bottom of the receiving cavity of said humeral cup base, said protrusions are evenly spaced from each other.

19. The humeral modular component according to claim 18, wherein each of said protrusions include a free end having a hook for engaging the groove of said humeral cup base, said protrusions and/or said wall are sufficiently flexible to allow for insertion of the shell into said receiving cavity of said humeral cup base.

20. The humeral modular component according to claim 14, wherein said flat surface of said base includes a beveled or rounded interior edge and/or a beveled or rounded exterior edge on its top.

21. The humeral modular component according to claim 14, wherein said flat surface is inclined from its exterior periphery towards said curved cavity.

22. The humeral modular component according to claim 14, wherein the surface area of said curved cavity is greater than a surface area of said flat surface.

23. The humeral modular component according to claim 14, wherein said curved cavity has a variating surface with a non-uniform level of curvature over the surface of said curved cavity or said curved cavity is concave.

24. (canceled)

25. The humeral modular component according to claim 14, wherein said humeral cup base is made from Co—Cr and said shell includes polyethylene or a related articulating material or Vitamin E HDPE.

26. (canceled)

27. A modular shoulder prosthesis system comprising:
a baseplate having
a base with a plurality of attachment holes passing therethrough, and
a central stem extending from a bottom surface of said base and axially centered with one of said plurality of attachment holes;
a modular component configured to be removably attached to said base, said modular component including a glenosphere with an optional inferior tilt of approximately 10 degrees;
a humeral base that is either a humeral stem or a second baseplate, said humeral base having a receiving cavity extending in from one face; and
a humeral modular component configured to cooperate with said modular component, said humeral modular component according to claim 14.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. A modular component for use in a modular shoulder prosthesis system having a baseplate with or without notches and/or attachment points, the modular component comprising:
a base having a cavity configured to receive a hemispheric humeral head or a prosthetic replacement of same, and said cavity is framed by a flat surface, and
a plug extending from a surface of said base opposite said cavity.

45. (canceled)

46. The modular component according to claim 44, wherein said flat surface is inclined from its exterior periphery towards said cavity.

47. The modular component according to claim 44, wherein a surface area of said cavity is greater than a surface area of said flat surface.

48. (canceled)

49. The modular component according to claim 44, wherein said base having a beveled or rounded interior edge around said cavity and/or a beveled or rounded top exterior edge.

50. The modular component according to claim 44, wherein said cavity is a variating surface with a non-uniform level of curvature over its surface or said cavity includes a concave surface.

51. (canceled)

52. The modular component according to claim 44, wherein said plug is a Morse taper central plug.

53. The modular component according to claim 44, wherein said modular component includes Cobalt-Chromium, TiN coated Ti5Al4V, or metal.

54. (canceled)

55. A modular shoulder prosthesis system comprising:
a baseplate having
a base with a plurality of attachment holes passing therethrough, and
a central stem extending from a bottom surface of said base and axially centered with one of said plurality of attachment holes;
a modular component configured to be removably attached to said base, said modular component according to claim 44;
a humeral base that is either a humeral stem or a second baseplate, said humeral base having a receiving cavity extending in from one face; and
a humeral modular component configured to cooperate with said modular component, and
wherein said any baseplate is capable of attachment to different modular components and said humeral base is capable of attachment to different humeral modular components to facilitate a future reverse total shoulder replacement with a change in the modular component and change in the humeral modular component.

56. (canceled)

57. (canceled)

58. (canceled)

59. (canceled)

60. (canceled)
